(19) United States
(12) Patent Application Publication
(10) Pub. No.: US 2013/0178915 A1
(43) Pub. Date: Jul. 11, 2013

(54) HIGH POWER ULTRASOUND WIRELESS TRANSCUTANEOUS ENERGY TRANSFER (US-TET) SOURCE

(71) Applicant: PIEZO ENERGY TECHNOLOGIES, LLC, Tucson, AZ (US)

(72) Inventors: Leon J. Radziemski, Tucson, AZ (US); Inder Raj Singh Makin, Mesa, AZ (US)

(73) Assignee: PIEZO ENERGY TECHNOLOGIES, LLC, Tucson, AZ (US)

(21) Appl. No.: 13/734,817

(22) Filed: Jan. 4, 2013

Related U.S. Application Data
(60) Provisional application No. 61/585,101, filed on Jan. 10, 2012.

(51) Int. Cl. A61N 1/378

(52) U.S. Cl. CPC A61N 1/3787
USPC 607/61

(57) ABSTRACT

A bio-implantable energy capture and storage assembly is provided. The assembly includes an acoustic energy transmitter and an acoustic energy receiver. The acoustic energy receiver also functions as an energy converter for converting acoustic energy to electrical energy. An electrical energy storage device is connected to the energy converter, and is contained within a bio-compatible implant for implantation into tissue. The acoustic energy transmitter is separate from the implant, and comprises a substantially 2-dimensional array of transmitters.

### CROSS REFERENCE TO RELATED APPLICATION

This application claims priority from U.S. Provisional Application Ser. No. 61/585,101, filed Jan. 10, 2012, the contents of which are incorporated herein in their entireties.

### STATEMENT REGARDING FEDERALLY SPONSORED RESEARCH OR DEVELOPMENT

This invention was made in part with Government support under grants number 1R43EB007421-01A1 and R44EB007421 awarded by the National Institutes of Health (NIH). The Government has certain rights in the invention.

### BACKGROUND OF THE INVENTION

The present invention relates to systems for powering implanted devices. The invention has particular utility for systems for powering implanted devices such as heart-assist devices and will be described in connection with such utility, although other utilities are contemplated.

The present invention addresses a critical barrier to a major increase in the availability of heart assist-devices to patients in need: the present method of providing power to these devices. Many publications cite the shortcomings of the existing method, which uses percutaneous links to provide electrical power to Mechanical Circulatory Support Systems (MCSS), for example, left- or right-Ventricular Assist Devices (VADs). The percutaneously placed wires are sources of infection, they periodically break, and they limit the life style of patients because of measures they must take to avoid infections. A 2001 news release from NIH about the 1998-2001 REMATCH clinical study of percutaneously powered LVADs cited the probability of infection within 3 months of implantation to be 28%. As a result, at this time, the use of VADs is limited to bridge-to-transplant patients, those with extreme loss of heart capability. Wireless Transcutaneous Energy Transfer (TET) across tissue is the much-preferred, less-invasive method of providing power to these devices. The impacts of a TET power system are that it 1) overcomes a major disadvantage of the present percutaneous method of providing power, namely high susceptibility to infection, opening up a life saving technology to hundreds of thousands who suffer from heart failure, and 2) supports the increased use of presently implanted heart-assist devices, and 3) fosters new devices targeted to improving human health.

Powering of MCSS completely by implanted batteries, whether primary or secondary, is not possible with the batteries available today because of the continuous high power requirement, which in turn dictates a large and heavy battery. A TET system would deliver power directly to the application, while also charging an implanted battery which could take over for periods of 1 to 2 hours. Over the past 50 years much effort has been expended in trying to make an electromagnetic method of TET (EM-TET) work. U.S. Pat. No. 6,579,315, to Weiss discloses an EM-TET system for an artificial heart. U.S. Pat. No. 5,630,836 to Prem discloses an EM-TET system for both an artificial heart and a ventricular assist device. Papers (Mehta et al., 2001; Schuder, 2002; Slaughter and Myers, 2010; Danilov, 2010) disclose parts of an EM-TET system and even some clinical trials. However a device based on this principle is still not in the marketplace. Major issues that hold back EM-TET adoption include 1) heating of tissue due to misalignment of transmitter and receiver coils which expose metal to magnetic and electric fields that cause eddy-current heating, 2) heating due to losses in the coils, 3) loss of transmission efficiency with depth of penetration, due to decreased coupling of transmitter and receiver, and 4) decoupling due to perturbation of the inductance of the coils when they interact with nearby metallic or magnetic materials.

In my earlier U.S. Pat. No. 8,082,041 I describe an UltraSound TET (US-TET) system suitable for providing power to devices such as neurostimulators or pacemakers, primarily to recharge implanted batteries. My aforesaid patent also contains a description of the prior art with regard to ultrasound power transmission, which by reference is included here. These applications typically require a few Watts of input power, and typically less than a half-Watt of power at the application. Specifically my aforesaid patent teaches a bio-implantable energy capture and storage assembly, including an acoustic energy transmitter for contact with the skin, and an acoustic energy receiver converter for converting acoustic energy to electric energy; and a battery or capacitor connected to the energy converter, and a method of cooling the assembly. The acoustic energy receiver/converter, which preferably employs ultrasound, is contained within a biocompatible implant. Significant advantages of employing ultrasound include non-mechanical alignment, eliminating tissue heating due to electromagnetic effects, and delivering power across thicker segments of tissues.

The application of US-TET to providing high power for heart-assist devices, requires an order of magnitude more power than the aforementioned applications, typically at this time 10 Watts, or even 20 Watts or more at the device to be powered. These levels of power require new and novel approaches. The invention described here is a modality for transferring energy at a high rate (e.g. power) wirelessly and safely across the skin in quantities sufficient to directly power energy-intensive implantable medical devices.

There are few prior references to using ultrasound as a carrier of energy at the levels needed for heart assist devices. Suzuki, et al (2003) describe a hybrid magnetic-ultrasonic device that employs magnetostrictive materials to generate the pressure waves that carry energy across the skin. That paper mentions ultrasound, but refers to a different and more complex system that only demonstrated ˜5 W of output power. Lawry et al., (2010), cite a linear delivery of 81 Watts through thick steel via ultrasound at 1 MHz, with power transfer efficiencies of up to 55%.

An important advantage of US-TET is the ability to mitigate the effects of lateral and angular misalignment by non-mechanical means, leading to a completely self-aligning system that does not require patient intervention. Also, the ultrasound beam, in the near field, does not diverge significantly, hence losses due to depth of the implant are minimal. Both of these advantages accrue to ultrasound because of its wave nature, and the fact that for power transfer, the ultrasound wavelength at useful frequencies is much smaller than the dimensions of the ultrasound transducers. In EM-TET the converse is true, ruling out the use of the techniques described below.

It is thus an object of the present invention to provide new and novel wireless power transfer techniques which alleviate distress, pain, complications, and operations associated with infections suffered by patients who would instead have to use the present method of power delivery to heart assist devices.

### SUMMARY OF THE INVENTION

The present invention provides a method and apparatus for powering an implanted device, such as a heart-assist device, and more particularly to an ultrasound wireless Transcutaneous Energy Transfer (US-TET) source to power an implanted device. In one aspect, an external transducer is connected to a battery-driven controller that modulates the power provided to the transmitter. In another aspect, the power may be supplied by other means, for example from an electrical power outlet fixed in a home or any other location. The external controller will receive several feedback signals wirelessly from the implant in order to regulate the transmitter power and frequency, to stabilize the power provided to the MCSS at an adequate level, and provide peak power as necessary.

The ultrasound-transducer transmitter is fixed to the skin of the patient by one of several possible methods, with an air-excluding pad, an ultrasound coupling gel between the transmitter and the patient's skin, or an air excluding boot. If cooling is necessary to keep the temperature of the patient's skin and intervening tissue within safe bounds, a cooling device such as a circulating-liquid heat exchanger, one or more Peltier coolers, miniature high-capacity fans, or other methods can be attached to or nearby the transmitter assembly. Temperature sensing devices within the transmitter and receiver may be provided to relay temperatures to the external controller, which will then apply the correct power to the cooling device in order to keep the temperature of the transmitter, receiver, and intervening tissue at safe values. The piezoelectric elements which are the heart of the transmitter and receiver may be monolithic elements, or a one- or two-dimensional array of small piezoelectric elements. Capacitively Machined Ultrasound Transducers (CMUTs) or other mechanisms for inducing ultrasound vibrations are an alternative to conventional piezoelectric elements. In one embodiment, a 2-dimensional array can be used to provide non-mechanical alignment of transmitter and receiver in response to optimization signals generated within the implant and relayed back to the transmitter.

The ultrasound receiver is contained within an implantable case, the external surface of which is completely fabricated of biocompatible material. It may be implanted at a functionally appropriate distance below the skin surface, e.g. from about 10 mm to about 50 mm below the skin surface, or some distance larger, between, or smaller than those distances. The front, flat face of the implant is fixed in the tissue approximately parallel to the front, flat face of the transmitter. In another embodiment, curved faces are used to enhance focusing effects that optimize the power transfer. Within the case are components for wireless communication with the external controller, electronics for converting the ultrasound to electrical power, sensors for monitoring the temperature at various points within the implant, sensors for monitoring and obtaining the optimum conversion efficiency, and output devices to 1) an implanted battery and 2) directly to the implanted MCSS.

There are two geometrical issues affecting alignment of a transmitter over a receiver in both the electromagnetic and ultrasound methods. The first is lateral translation over the implant, and the second is angular misalignment between the transmitter and receiver. The use of an array transmitter enables compensation for both of these misalignments. The voltage, current and/or power out of the receiver is a signal fed back to the external controller which commands the array transmitter to search for the optimum alignment. In another embodiment, an imaging ultrasound system is added to the transmitter unit to provide the feedback on the depth and orientation of the implanted receiver, thereby assisting alignment.

In one aspect of the invention there is provided a bio-implantable energy capture and storage assembly, comprising: i. an acoustic energy transmitter and an acoustic energy receiver, said acoustic energy receiver also functioning as an energy converter for converting acoustic energy to electrical energy; ii. an electrical energy storage device connected to said energy converter, wherein said acoustic energy receiver-converter is contained within a biocompatible implant for implantation in tissue, wherein said acoustic energy transmitter is separate from said implant.

In one embodiment the transmitter is comprised of a monolithic disc. In another embodiment of the invention the transmitter is comprised of a 2-dimensional array of elements arranged on a support.

In another embodiment the substantially 2-dimensional array of elements is arranged in a circle.

In one embodiment the substantially 2-dimensional array of elements is arranged in a substantially regular 2-dimensional geometric shape, such as, by way of example, without limitation, a square, a pentagon, a hexagon and an octagon.

In one embodiment the bio-implantable energy capture and storage assembly will include a wireless feedback loop between the implant and transmitter, for monitoring one or more parameters related to an output power of the receiver.

In another and preferred embodiment, the bio-implantable energy capture and storage assembly further includes a device for cooling the energy transmitter and tissue.

In one embodiment the bio-implantable energy capture and storage assembly further includes sensor transmitters and receivers on the acoustic energy transmitter, connected in said a feedback loop. In such embodiment, the sensor transmitters and receivers preferably comprise ultrasonic elements.

In a second embodiment the invention also provides a bio-implantable energy capture and storage assembly, comprising: i. an acoustic energy transmitter and an acoustic energy receiver, said acoustic energy receiver also functioning as an energy converter for converting acoustic energy to electrical energy; ii. an electrical energy storage device connected to said energy converter; and iii. a device for providing conditioned power directly to a load, connected to said energy converter, wherein said acoustic energy receiver-converter is contained within a biocompatible implant for implantation in tissue, wherein said acoustic energy transmitter is separate from said implant.

In such second embodiment the transmitter is comprised of a 2-dimensional array of elements arranged on a support.

In such second embodiment the substantially 2-dimensional array of elements is arranged in a circle.

In such second embodiment the substantially 2-dimensional array of elements is arranged in a substantially regular 2-dimensional geometric shape, such as, by way of example, without limitations, a square, a pentagon, a hexagon and an octagon.

In such second embodiment, the bio-implantable energy capture and storage assembly further includes a wireless feedback loop between implant and transmitter units for monitoring one or more parameters related to an output power of the receiver.

In such second embodiment the bio-implantable energy capture and storage assembly also preferably further includes a method of cooling the energy transmitter and tissue.

In such second embodiment the bio-implantable energy capture and storage assembly further includes sensor transmitters and receivers on the acoustic energy transmitter, connected in said feedback loop.

In such embodiment the sensor transmitters and receivers preferably comprise ultrasonic elements.

In still yet another embodiment there is provided a process for optimizing a position of the bio-implantable energy capture and storage assembly as described which comprises positioning the assembly on a patient, measuring receiver output, repositioning the assembly and again measuring receiver output, repeating repositioning the measuring, each time repositioning the assembly a smaller amount until changes in repositioning no longer yield perceivably higher power.

### BRIEF DESCRIPTION OF THE DRAWINGS

Further features and advantages of the present invention will be seen from the following detailed description, taken in connection with the following detailed description, wherein like numerals depict like parts, and wherein:

FIG. 1 is a schematic of the system of the present invention;

FIG. 2A is a block diagram of the components contained within the external controller part of the invention;

FIG. 2B is a block diagram of the components contained within the transmitter assembly part of the invention;

FIG. 2C is a block diagram of the components contained within the implant assembly part of the invention;

FIG. 3 is a schematic of the transmitter, tissue, and receiver part of the system of the invention;

FIG. 4 is a schematic of an ultrasound transducer used in the present invention;

FIG. 5 illustrates the efficiency of ultrasound power transmission as a function of frequency in accordance with the present invention;

FIGS. 6A-6D show lateral alignment of an array transmitter and receiver in accordance with the present invention;

FIG. 7A illustrates ultrasound beam turning using an array of transmitters with a phase difference imposed between the elements of the array in accordance with the present invention;

FIG. 7B illustrates the desensitization of power delivery to angular misalignment as the number of elements in a linear array increases, for transducers of 25 mm diameter and a frequency of 1 MHz in accordance with the present invention;

FIG. 7C illustrates the desensitization of power delivery to angular misalignment as the number of elements in a linear array increases, for transducers of 75 mm diameter and a frequency of 1 MHz in accordance with the present invention;

FIG. 8 illustrates the effect of cooling on the temperature at the face of the implant in accordance with the present invention;

FIG. 9 is a schematic block diagram of the components of the wireless communication link in accordance with the present invention.

### DETAILED DESCRIPTION

### Overall Assembly

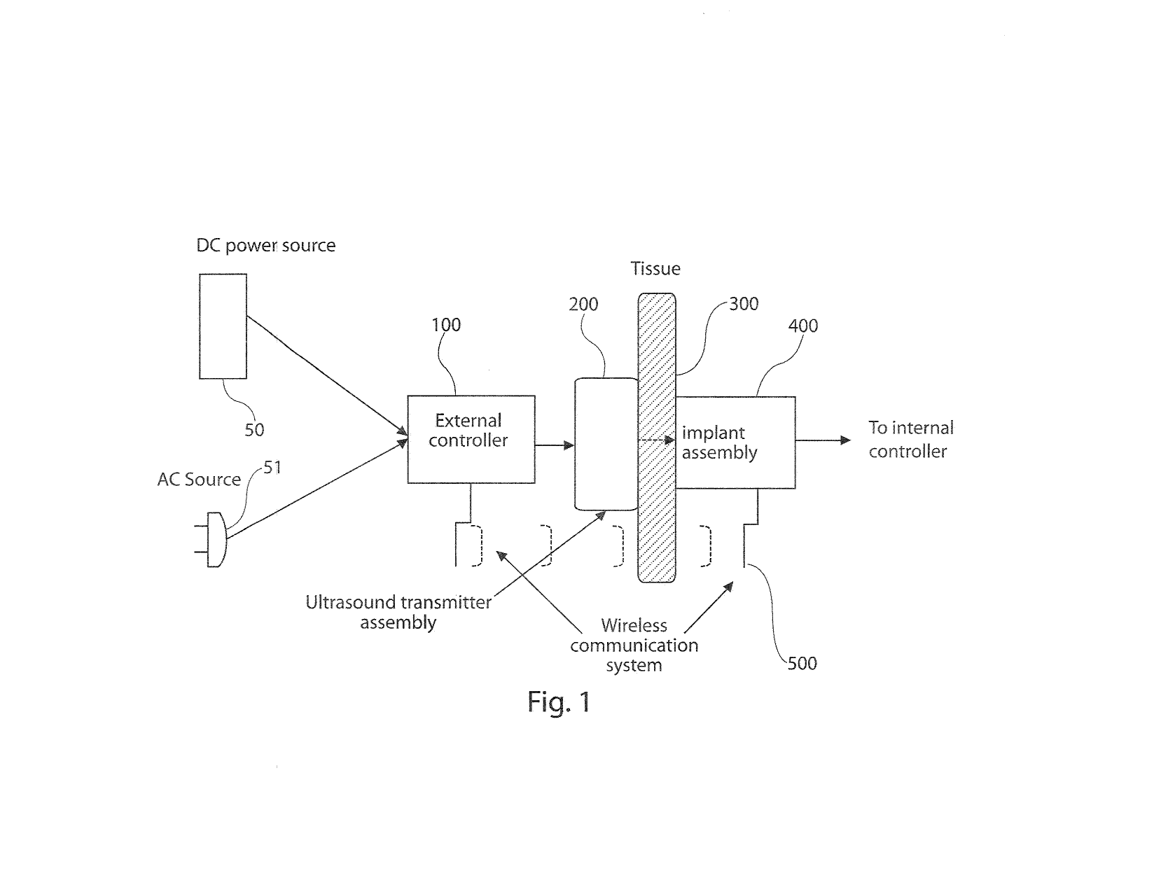

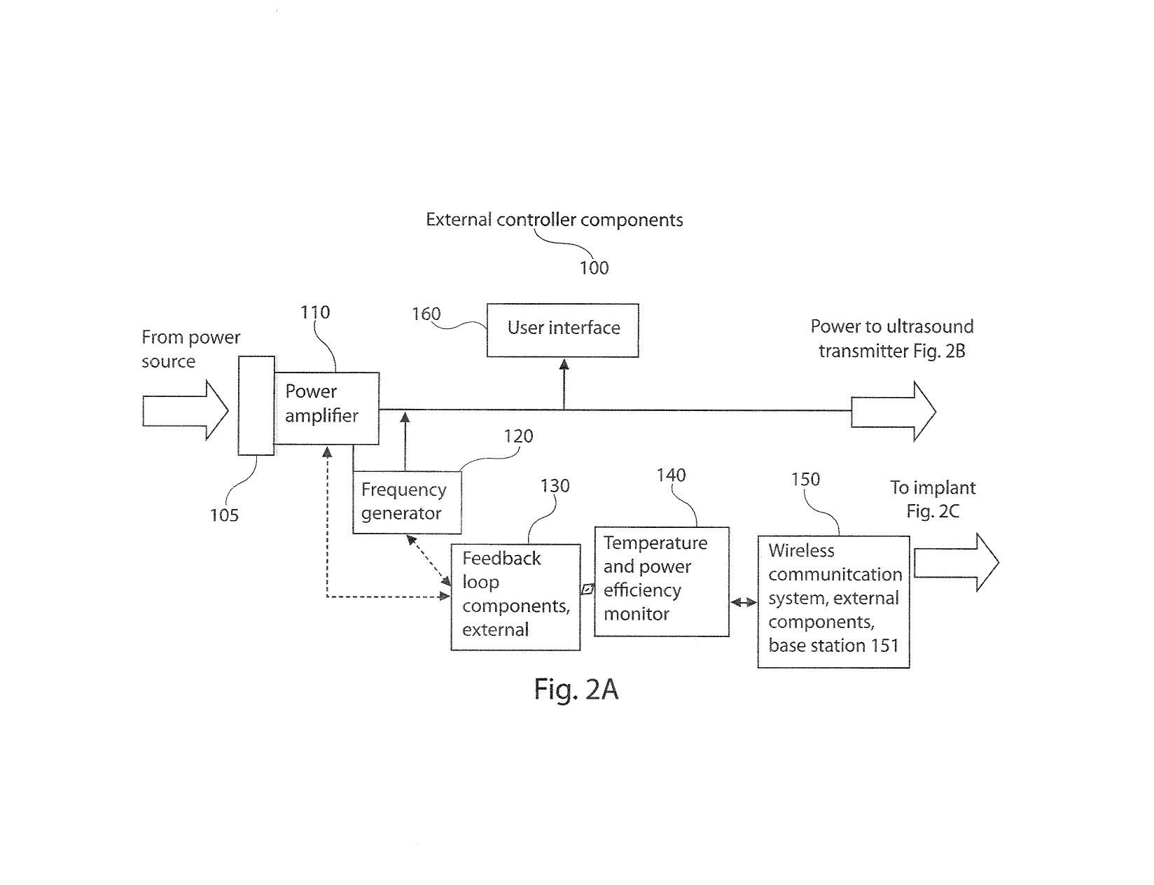

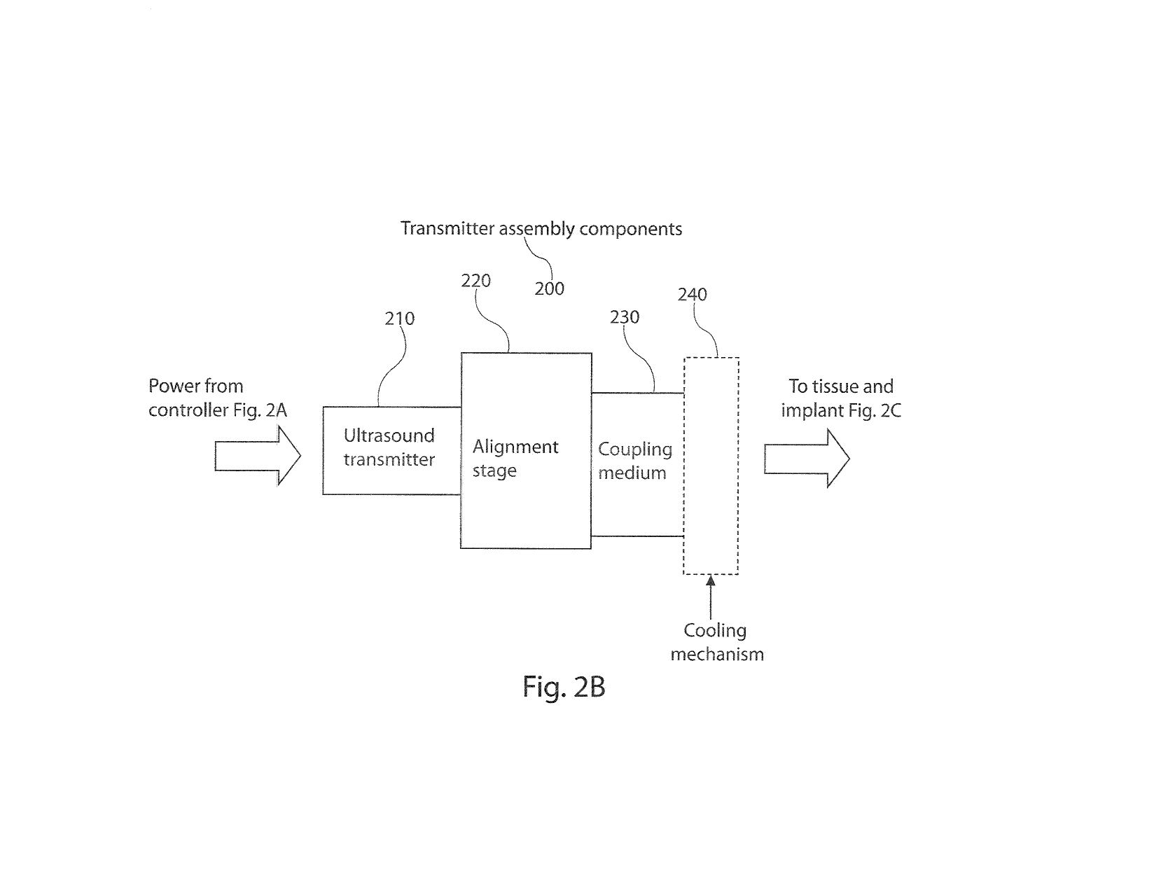

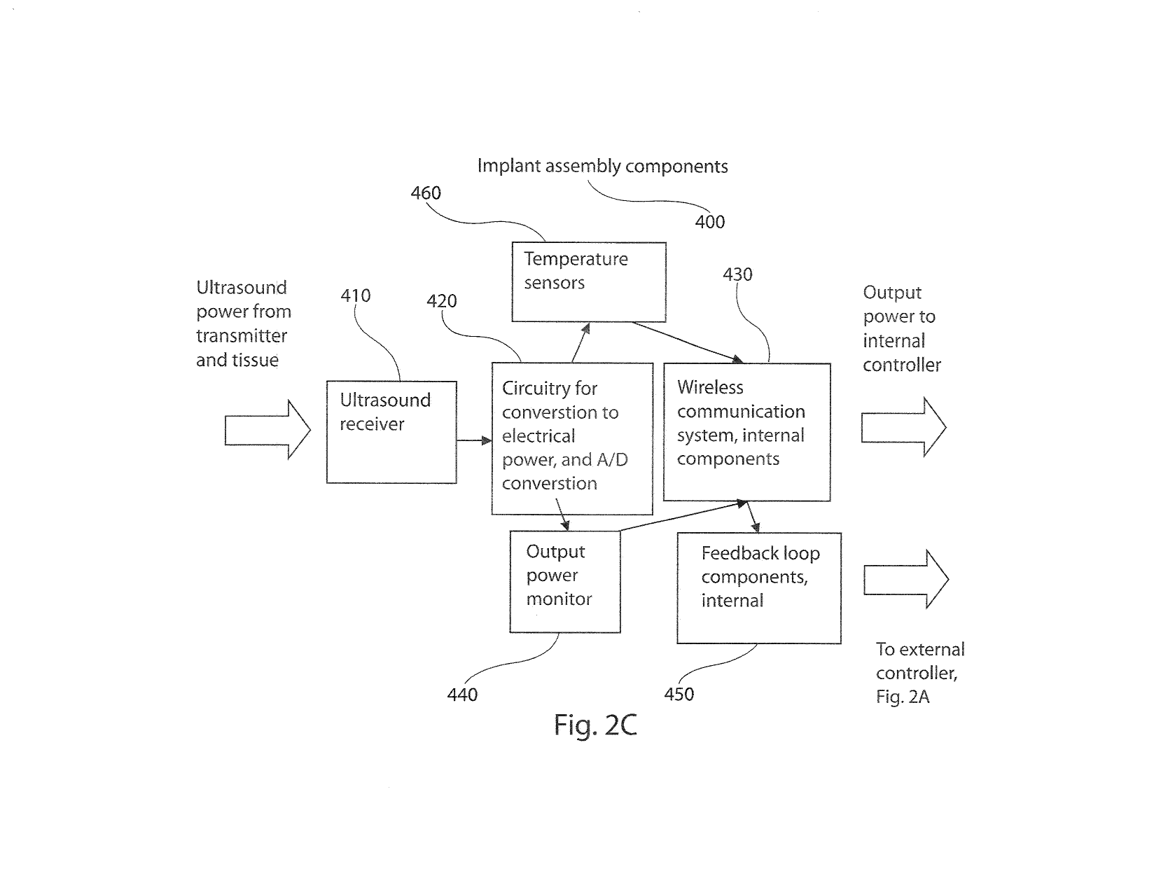

FIG. 1 is an overall block diagram of an US-TET system in accordance with the present invention. FIGS. 2A, 2B, and 2C are block diagrams the items within the external controller 100, the transmitter assembly 200, and the implant assembly 400. Referring to FIG. 1, two possible sources of power can operate the system. They are either a direct current (DC) power supply 50 such as a battery, typically worn by the patient, or a conventional room alternating current (AC) source 51. Circuitry within the external controller 100 determines whether the input power is low frequency AC. If so, it proceeds through a DC converter and then through circuitry 120 which converts it to high frequency ultrasound. The external controller 100 determines the level of input power and frequency of the ultrasound. These can be operated in two modes, manually and automatically, the latter via a feedback loop 130 and 450 made possible by the wireless communication system 500, which has external 150 and internal 430 components. The output of the external controller 100 is connected to the transmitting transducer 210, which is disposed adjacent to the skin of the subject. After transmission through human tissue 300 the ultrasound is incident on the receiver 410, which is disposed on or under the face of the implant 400 adjacent to internal tissue.

After conversion back to electrical power via circuitry 420 residing within the implant 400, the power is directed to an implanted controller which modulates the current and other sensors for the operation of the MCSS, and as necessary, to replenish an internal DC source such as a battery. The internal battery is used to power the MCSS for short periods of time such as a few hours, while the patient removes the external supply to bathe or for other conveniences. A wireless communication system 500 between the external controller and the implant, such as a Zarlink 405 MHz medical-band system, provides a means of monitoring functions of the receiver and implant, issuing performance commands to the elements within it, and maintaining one or more feedback loops 130 and 450 for optimization of performance.

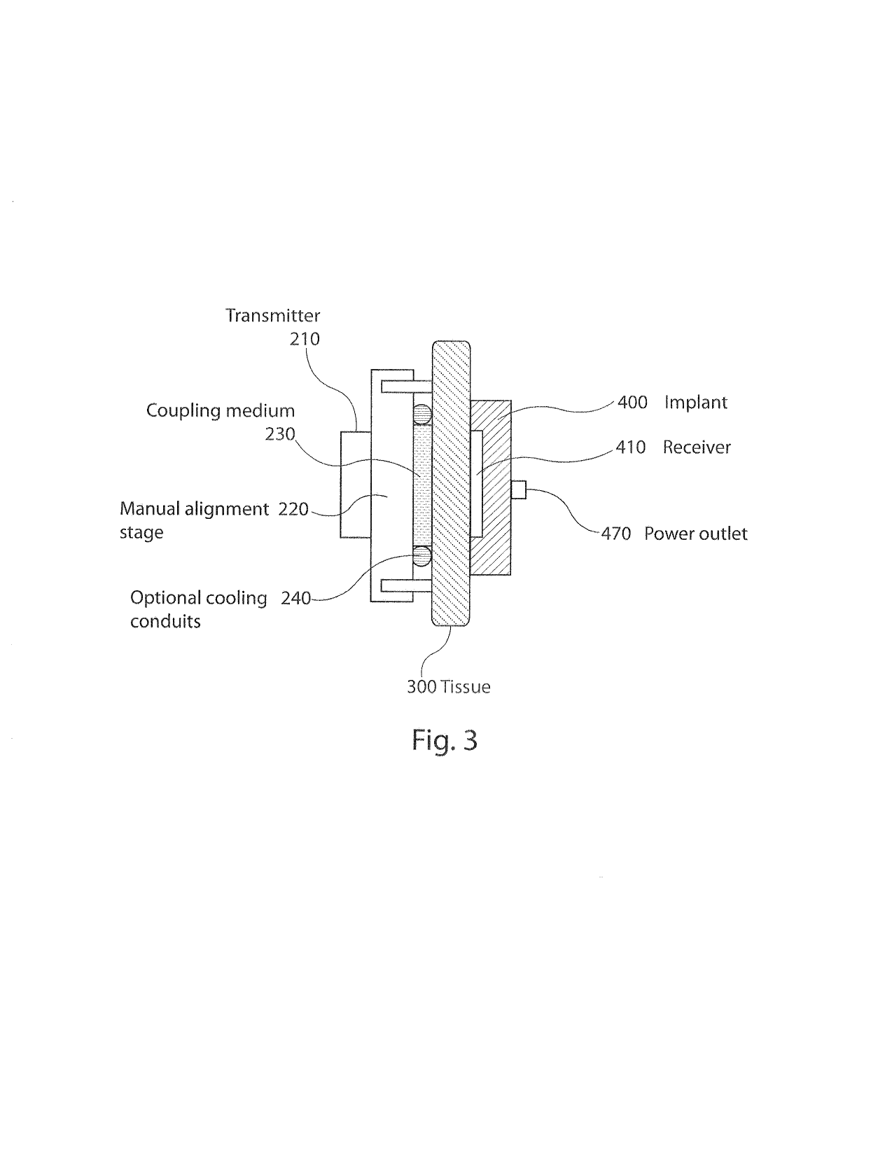

FIG. 3 shows a schematic arrangement of the transmitter-tissue-implant part of an US-TET system. The transmitter transducer 210 transmits acoustic energy which may be continuous or pulsed with a variable duty cycle, via sine waves, square waves, triangular waves or an arbitrary repetitive shape, wirelessly through an external coupling medium 230 which may be a gel pad, or ultrasound coupling pad, or some other air-excluding medium. Essentially all air preferably will be excluded, between the skin of the patient and the ultrasound transmitter, since air strongly attenuates ultrasound over frequencies of 100 kHz. A cooling system 240, if needed, may be deployed as schematically shown. Sufficient external cooling has been observed to penetrate the dermis, cooling the intervening tissue and the implant as well. After penetrating the epidermis, dermis, and possibly fat and muscle layers, the ultrasound is incident on a biocompatible implanted container 400 which has the receiver 410 on or against the inside of the front face, and other elements packaged within it. The receiver transducer 410 converts the acoustic to electrical energy. This energy proceeds via the schematically shown power outlet 470, which leads to the internal controller, power conditioning circuitry, and then to an application such as the MCSS.

### Transmitter and Receiver Ultrasound Transducers

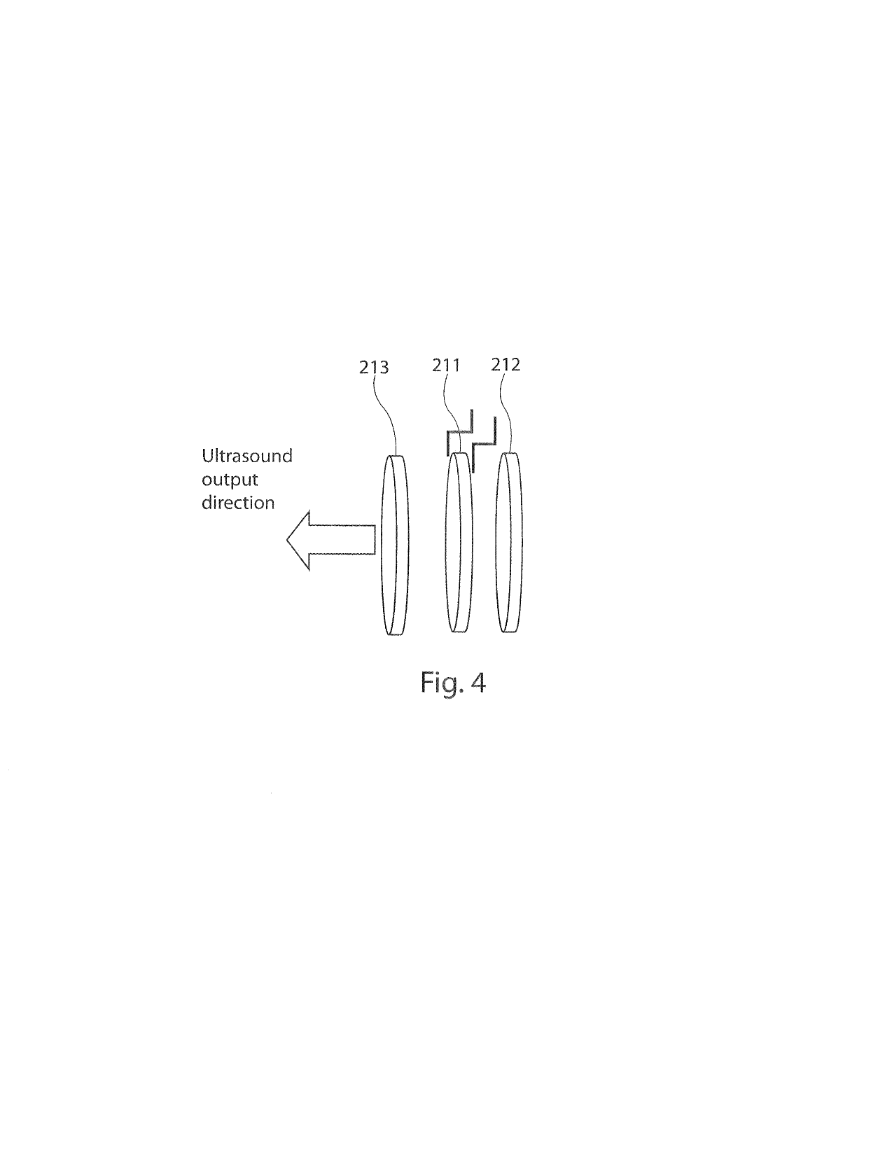

FIG. 4 shows an exploded view of one embodiment of an ultrasound transducer, a device which converts electrical energy to vibrational energy, and vibrational energy to electrical energy useful in the present invention. In its simplest form it is comprised of a piezoelectric material which changes its dimensions when an electric field is placed across it. Other materials that respond to fields may be used, such as magnetostrictive materials. In one embodiment, a piezoelectric disk 211 comprised of a ceramic matrix in which are embedded crystals of Lead-Zirconium-Titanate (PZT) can be the basis of a transducer. Other materials such as Lead-Magnesium-Niobate in Lead-Titanate (PMN-PT) may also be used. The two flat surfaces are coated with a conducting film to which electrodes are attached and which carry the electromagnetic wave to the material, causing it to shrink or expand slightly at the frequency of the wave. The disk 211 normally has a backing 212 to augment the conversion, and is housed in a case made of plastic or aluminum or titanium or other material. In another embodiment, the disk 211 is bonded directly to the inner face of a titanium implant case 400 which contains all the components of the implanted device, and which is hermetically sealed. The element 213 between the disk and the medium through which the vibrations are passing has a thickness such as to minimize the reflection of the wave, typically a quarter or full wave thick, and possibly comprised of multiple layers.

The transmitter 210 and receiver 410 transducers may have a high-Q (narrow bandwidth) and be designed and manufactured to have closely matched resonance frequencies. In a second embodiment, one of the units may have a high-Q resonant frequency and the other a lower-Q wider bandwidth resonance, making the combination less sensitive to temperature-induced changes of frequency in either unit. In a third embodiment, both units may have a lower-Q and wider bandwidth. Although power requirements will dictate size and mass of the receiver, these can be quite small. A thin circular-disc piezo-receiver bonded directly to a titanium pacemaker face adds only 1.5 cm³ in volume and 7 g in mass. It is well known to those skilled in the art that maximum electrical or acoustic power is transferred between two objects when their electrical and acoustical impedances are matched (Woodcock, 1979). Optimization of the transducer impedances is accomplished with impedance matching software.

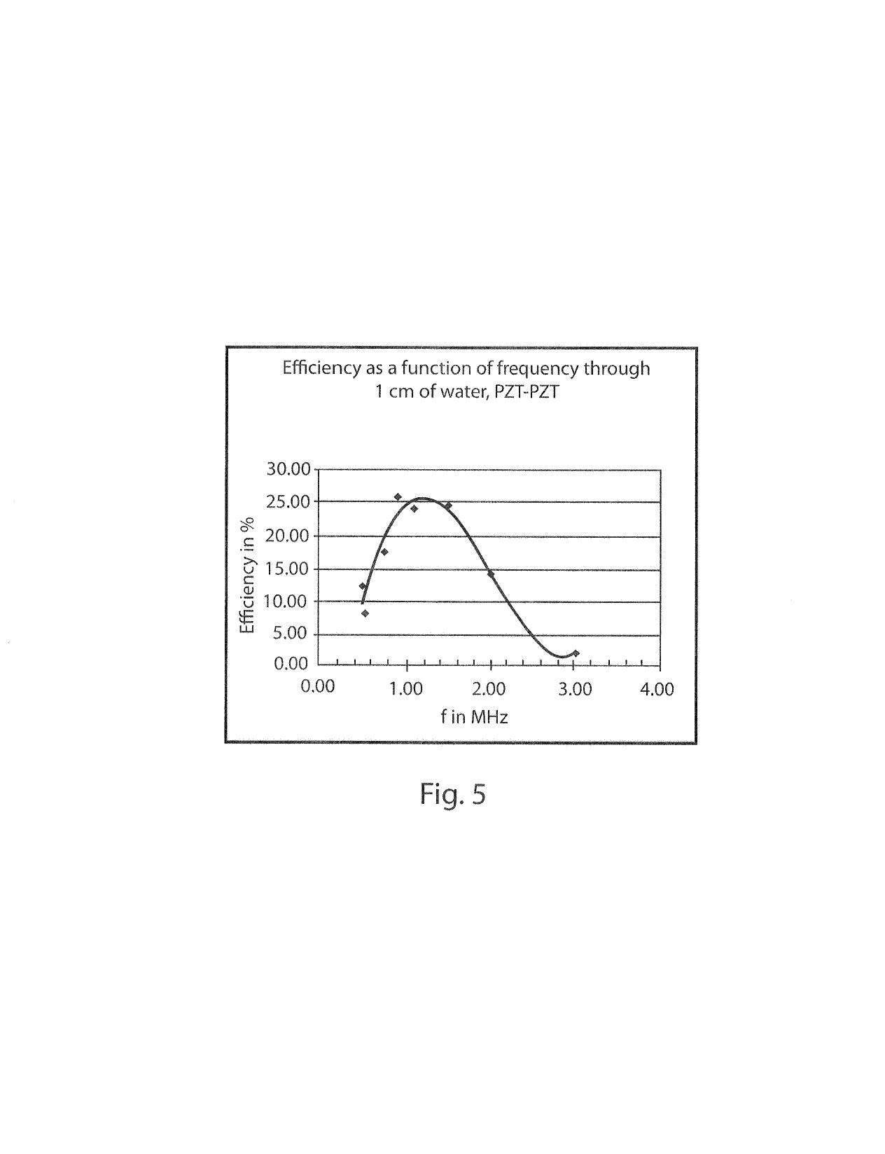

The frequency of the transducers is determined by a variety of constraints. At too low a frequency, below 500 kHz, there is the increased probability of cavitation which can lead to embolisms. At higher frequencies above 1 MHz, the absorption of tissue increases considerably, and the transducer element becomes quite thin. A series of experiments whose results are shown in FIG. 5 determined that an optimum frequency is in the range of 0.75 to 1.5 MHz. A value of approximately 1 MHz is an adequate compromise within the band. In addition to resonant frequency, the bandwidth is also an important transducer parameter. Too small a bandwidth, such as in the kilohertz range, can lead to a lack of overlap of the transmitter and receiver resonant frequencies due to differential heating of transmitter and receiver during operation, with a consequent loss of transmission efficiency.

### Design of Safe High-Power Transmitter and Receiver Transducers

A primary consideration in wireless transmission of power through tissue, whether it be electromagnetic or ultrasound, is the avoidance of tissue damage. There are well known guidelines to achieve this for ultrasound, keeping the acoustic intensity at the skin at or below a maximum of 0.7 W/cm² (AIUM, 1993; Hedrick, 2005; NCRP Report 113, 1992), a very conservative value adopted to avoid significant temperature rise in critical tissue structures including the fetus during obstetrical imaging. This dictates, for a given input electrical power, the minimum area of a transmitter that applies the power to a patient. An example calculation of the required transducer area follows. Assume a conversion efficiency of electrical to ultrasound power of 70%. Then 1 W/cm² electrical intensity would produce 0.7 W/cm² of acoustic intensity. Using conventional expressions for the relationship between ultrasound intensity and particle motion in water (analogous to soft tissue), at 0.7 W/cm², particle motion is calculated to be a maximum of 15 nanometers, a very small amount. Assume that 40% of the electrical power from the transmitter issues from the implanted receiver, and that 20 Watts is necessary at that point to operate the MCSS. That places a requirement of 50 Watts of electrical power at the transmitter, requiring a transmitter area of 50 cm² (diameter of 8 cm) to keep the acoustic intensity at 0.7 W/cm². An additional metric for device safety is that tissue temperature increase due to the TET system application be less than 2° C. That metric is met by having the power spread out over the large transducer areas. An alternate embodiment employs a cooling system.

The main non-thermal possibility for tissue damage arises from cavitation, rapid expansion and contraction of air bubbles, primarily in the lungs. The probability for this effect increases with ultrasound frequencies below 500 kHZ, and for locations where ultrasound can interact with lung tissue. Avoiding such locations and using a frequency around 1 MHz minimizes this possibility.

### External Controller

As shown in FIG. 2A, the external controller 100 contains a variety of components. When converting from input DC power, it goes through a DC to DC converter 105 to bring it to a range of useful current and voltage. It then proceeds to a signal generator 120 such as a variable frequency oscillator or a synthesized signal generator to condition it to the frequency of interest. When converting from input alternating current, which may be 120 V, 60 cycle or some other normally used combination, first the electrical power goes through an AC to DC conversion 105, and then follows the steps outlined above for a DC power source. In both cases the power at the appropriate ultrasound frequency then proceeds through an amplifier 110 to bring it to the level required for the application. The power level can be set manually by an input command, or be placed under the control of a feedback loop 130 and 450 which keeps it at the specified value. A useful feedback parameter, whose value is relayed from the implant to the external controller, is the output power from the ultrasound receiver. Typically it would be desirable to keep the output power stable for optimum operation of the application.

A second important function of the controller is to monitor and change the frequency of the ultrasound. Typically the range of changes are approximately 10% of the resonant frequency, and this is achieved via a variable frequency oscillator 120 or a synthesized signal generator 120, methods well known to those skilled in the art. The frequency can be set manually with an input command, or can be placed under the control of a frequency feedback loop 130 and 450.

Embedded in the controller is the antenna 150 which enables reception of communications from the implant on a medical communication band. These include receiving values of temperatures 140 being monitored in various implant locations, monitoring the efficiency of power conversion 140, and monitoring transmitter and receiver unit alignment. In one embodiment, a hybrid National Instruments Signal Express plus C++ code collects and stores the data automatically and continuously for up to 10 parameters, both for patient information on a user interface 160 and for periodic diagnostic downloading. The latter allows a variety of charts, comparisons, and figures of merit to be recorded and analyzed, to monitor the health of the system.

Software compares the temperature readings with a preset regime of safe temperatures and, if necessary, sends a warning to a user interface 160, similar to a smart phone, which allows the patient to monitor power efficiency and receive safety warnings. The user interface communicates with the controller using a wireless protocol, such as Bluetooth, Wi-Fi, or other advanced method.

### Transmitter Unit and Components

Power from the external controller 100, at an ultrasound frequency, proceeds to the transmitter assembly 200 and transmitter transducer 210. This activates the transmitter transducer 210 to convert electrical power to ultrasound for transmission through human tissue 300. The transmitter alignment stage 220 contains a method of being fixed to the patient, a manual adjustment method to align the transmitter and receiver faces, a non-mechanical adjustment method to align the wave front from the transmitter parallel to the receiver face, a space for an element 230 which excludes air between the ultrasound transmitter and the skin of the patient, and if necessary, a cooling method 240. The alignment stage may be fixed to the skin by means of a sticky tape on the bottom or over the top of the alignment stage (Mehta et al., 2001, FIG. 3). Another embodiment has a strap or holster in addition to or in place of the sticky tape to secure the transmitter unit to the skin. Another embodiment attaches the stage via a slight suction generated by a boot and clamp method, as used for affixing items to the inside of an automobile windshield. The manual adjustment method, in one embodiment, three screws of fine pitch set in a triangle, which aligns the platform angularly over the implant. Initial lateral alignment is performed over the slight protrusion of the implant which rises from a few millimeters to one centimeter or more over the adjacent tissue. A lightweight cone on the bottom of the alignment platform fits over the protrusion, ensuring secure lateral alignment.

### Implant Unit and Components

FIG. 2C is a block diagram of the components of the implant assembly. FIG. 3 illustrates the placement of the implant 400 connected to the tissue 300. The piezoelectric element 410 which is the key element of the receiver transducer, is placed on the front face of the implant 400, or underneath it and permanently affixed to it. Adjacent to that element is found circuitry 420 which converts the ultrasound to electrical power, AC or DC, as required by the application which is receiving the power. The converted power is monitored 440 and the analog data stored. Embedded in various locations in the implant will be thermal sensors 460 which enable the temperatures in those locations to be monitored. Circuitry for analog to digital conversion of those data 420 are also embedded in the implant, as are internal wireless communication components 430, including an antenna. The data so transmitted are the input for the feedback loop 130 and 450. The external controller 100 then resets parameters such as power, frequency, and alignment in order to stabilize the power provided to the internal application.

### Non-Mechanical Alignment of Transmitter and Receiver

Alignment of the transmitter and receiver is an important issue both in EM-TET and US-TET. Even though the transmitter unit may be affixed securely to the skin over the implant, it is possible that the implant could move slightly within the somewhat flexible tissue in which it is placed. Hence a method of both lateral and angular alignment in the post-implanting phase, is desirable and necessary. Furthermore, it is desirable that the methods of alignment not depend on the patient's intervention, because the system may be required to operate virtually 24/7, even when the patient is asleep. Ultrasound provides a method for non-mechanical alignment not available to EM-TET.

One dimensional arrays of ultrasound transmitter elements are well known to those skilled in the art. Their principal applications are for scanning an ultrasound beam in space to image structures in the body, and for non-destructive testing of materials and weld integrities. Two dimensional arrays have been made as well, and the technology is advancing to make inexpensive 2-D arrays (Ranganathan, et al., 2004; Fuller et al., 2009).

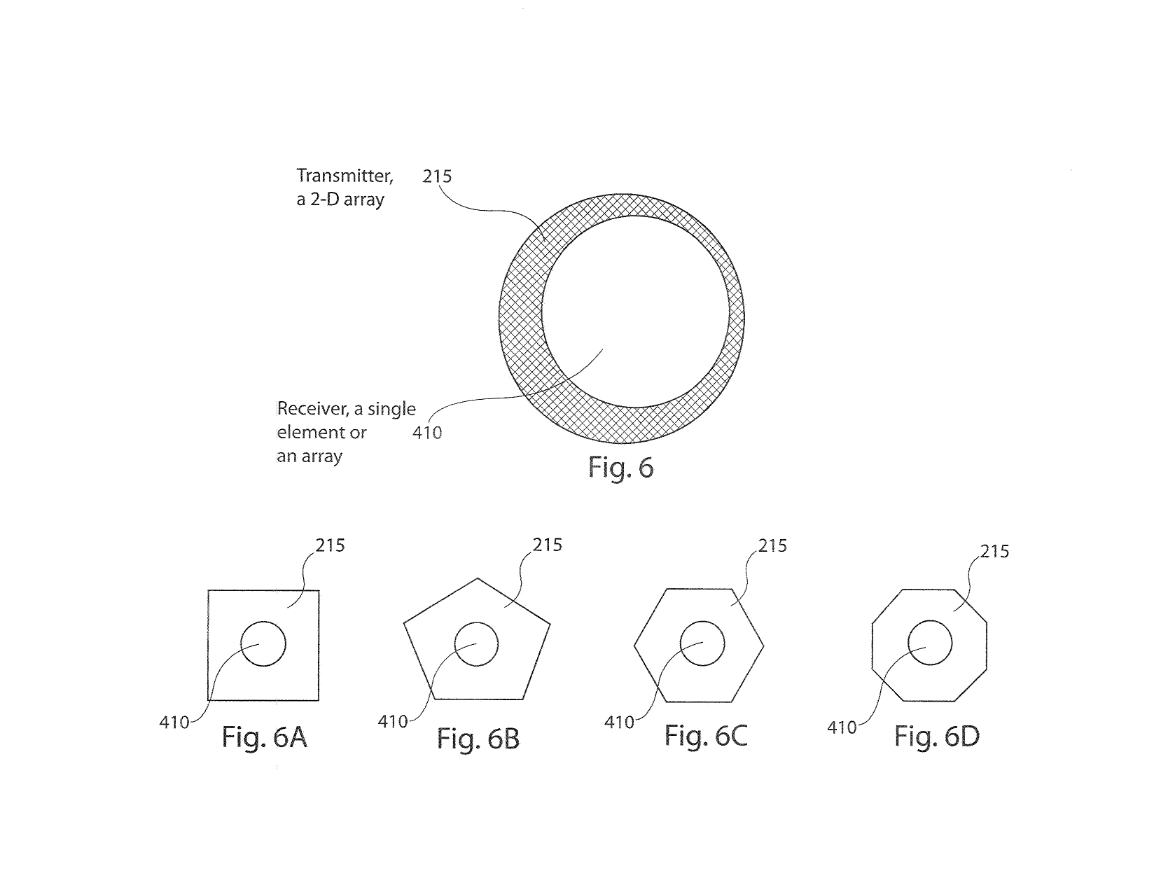

FIG. 6 shows an arrangement for lateral alignment of a larger substantially 2-D array 215 over a smaller receiver 410. Preferably the 2-D array of the transmitters is arranged on a circular disk, e.g. as shown in FIG. 6, although other regular 2-D geometric arrangements, e.g. square, pentagonal, hexagonal, octagonal, etc., shapes may be used as illustrated in FIGS. 6A-6D. For lateral alignment, a feedback loop 130 and 450 relays the output power level of the receiver back to the controller 100 that activates a number of elements in the 2-D array transmitter 215. The controller 100 activates elements sequentially along one axis, and then along a second axis perpendicular to the original direction. In this way the centroid of the active elements that maximizes or optimizes the output power is obtained. Once the optimum position is determined, the number of array elements that maximize or optimize the output power remain activated until a significant departure from the chosen output power is observed with the feedback loop 130 and 450, leading to a rescanning. The frequency of rescanning depends on the rapidity of changes in the lateral position, which is likely to be slow.

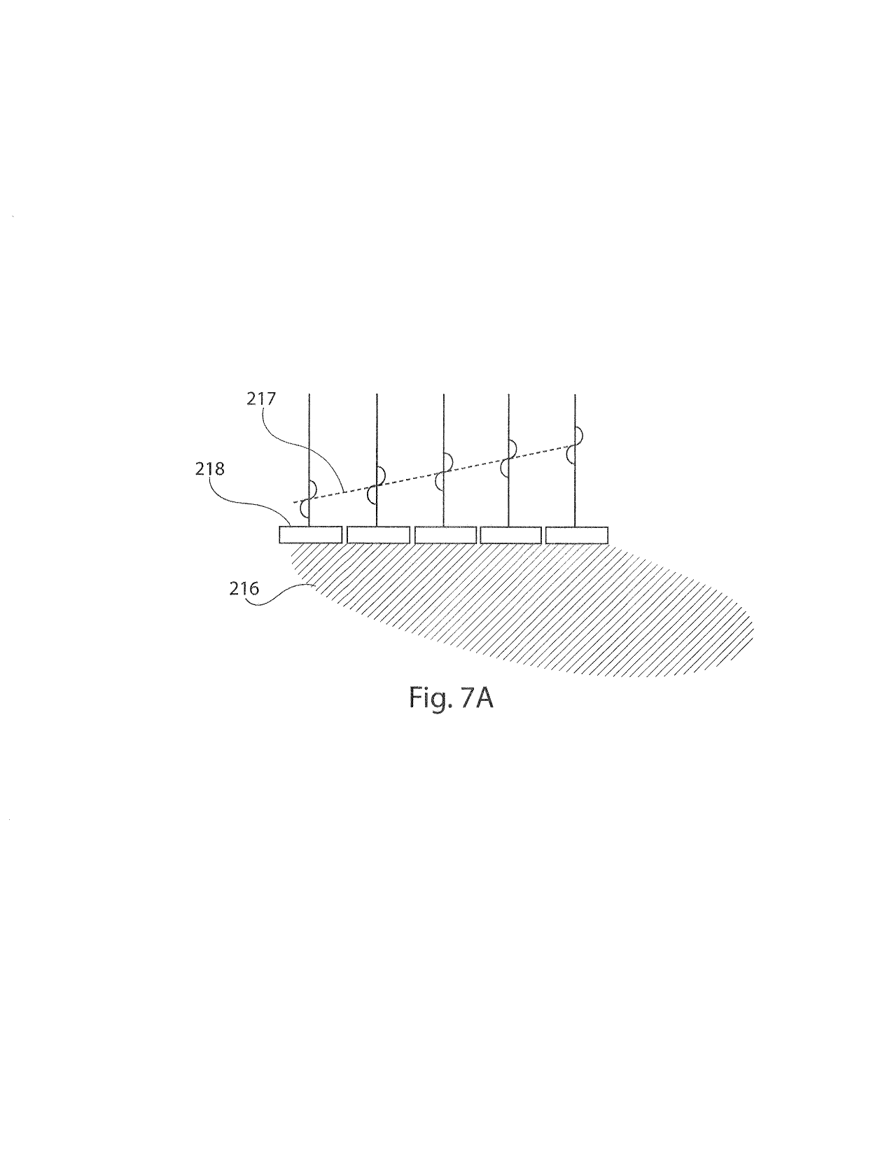

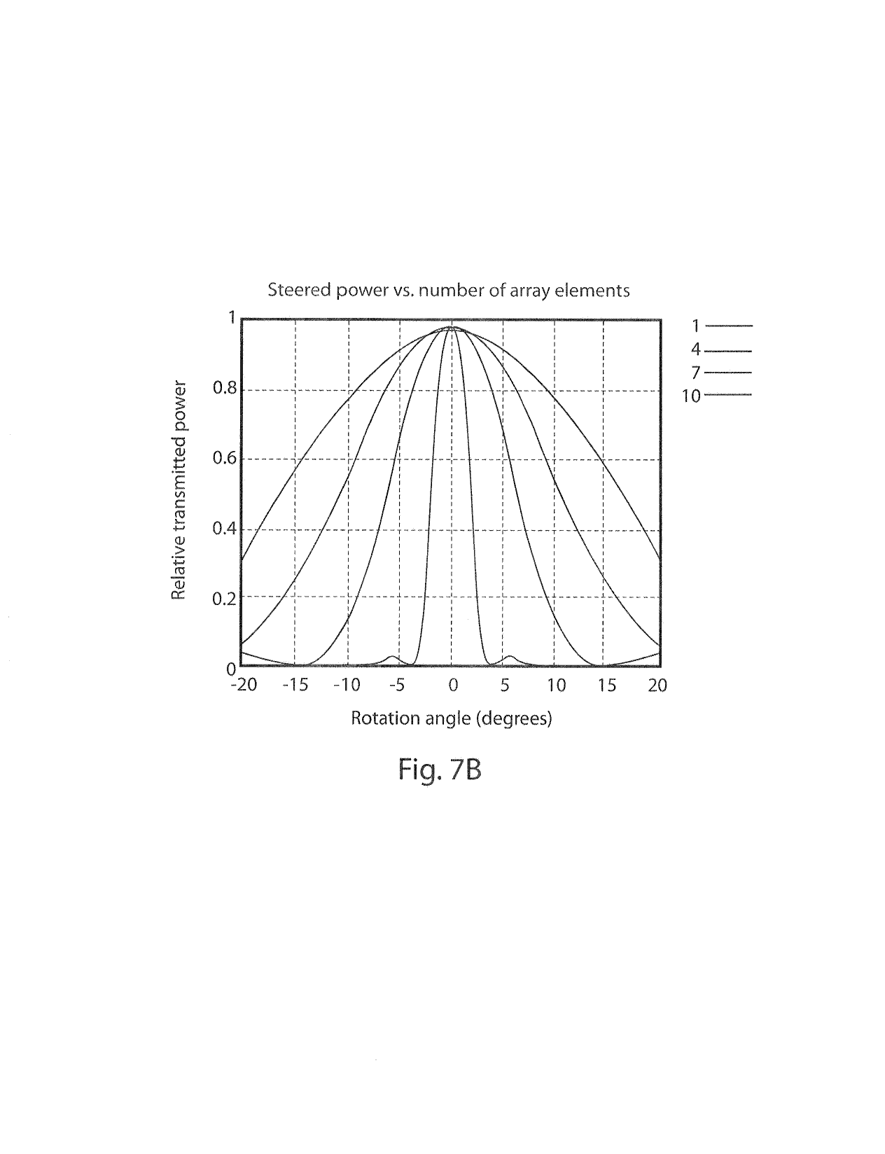

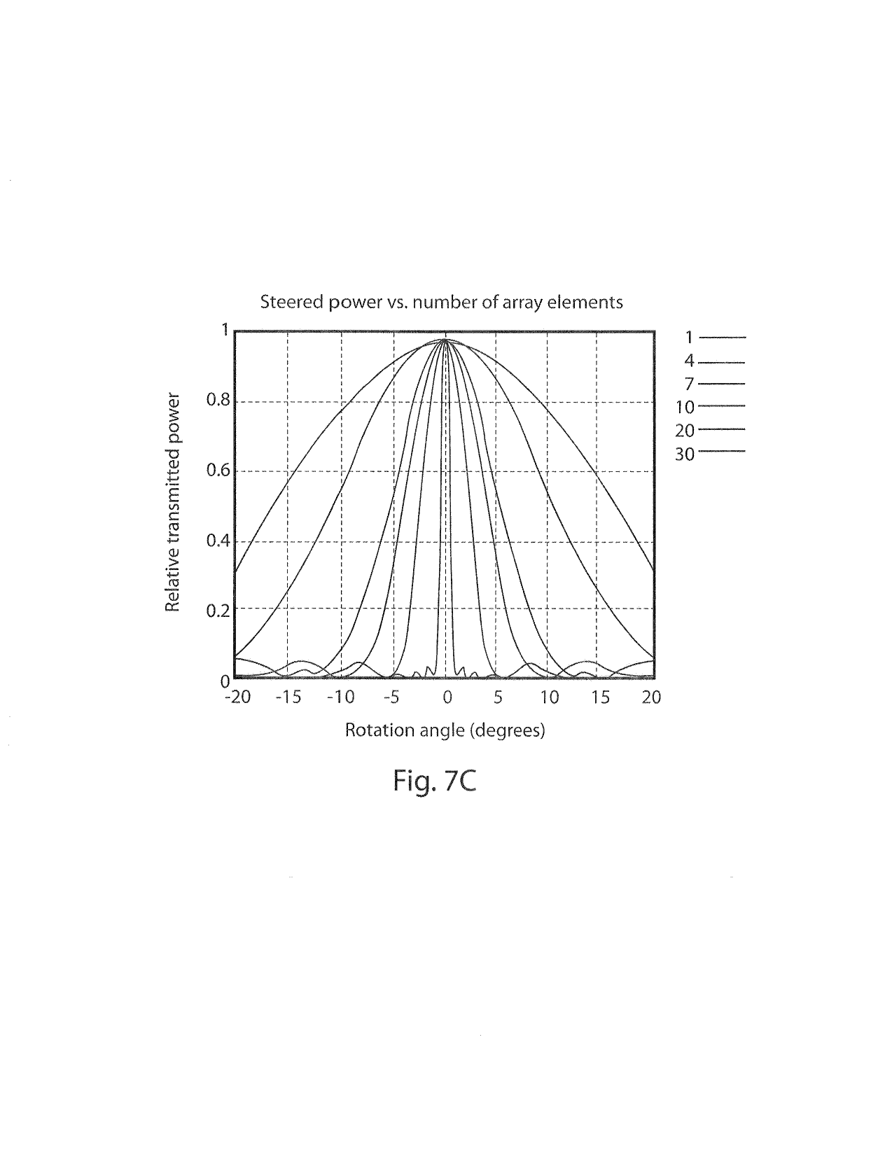

For angular alignment two effects are considered. The first of these is the turning of the beam wave front from parallel to the face of the transmitter array, through an angle that makes the wave front parallel to the face of the implanted receiver. This compensates for angular misalignment of the faces of the two transducers. For two dimensional surfaces this needs to be done along two axes. It is well known to those skilled in the art that this is accomplished by embedding a constant time differential, which results in a phase difference, between each element of the array. The result is shown schematically in FIG. 7A which illustrates the beam turning 216 by introducing a constant phase 217 between elements of a one-dimensional array 218.

The second effect deals with decreasing the sensitivity to alignment of two plane parallel transducers faces. Maximum power transfer takes place when the incoming wave is at the same phase at all points on the receiver. In order to keep the incoming wave from the transmitter in phase across the face of the receiver, the two must be aligned to within one-half wavelength. For a frequency of one MHz in tissue that is approximately 1 mm. This alignment condition becomes more and more stringent as the diameter of the transducers increase. For a 10 mm diameter transducer, the alignment condition is that the two surfaces be parallel to 1 mm out of 10 mm. For a 70 mm diameter transducer, the condition is 1 mm out of 70 mm. This condition is relaxed for an array because the width of the array element substitutes for the overall width of the whole array. An array element width can vary from 0.1 mm to several millimeters. This relaxation is shown in FIG. 7B in a model-based calculation result for an ultrasound frequency of 1 MHz. There is plotted the steered power versus the number of array elements for a pair of 25 mm diameter transducers, where the transmitter is a one-dimensional array, and the receiver a monolithic single element. The narrowest trace is for one element, then follow in increasing width the traces for 2, 3, and 4 elements. For a single 25 mm diameter transmitter element (the whole transducer), the power falls to 80% within a degree of misalignment on either side of the center line. Increasing the number of elements per unit area to 10 spreads the 80% power cone to ±8°. That in turn, reduces the restriction on the angular alignment to retain 80% power, to ±8°. FIG. 7C shows the result of a calculation for a 70 mm diameter transmitter array with up to 30 elements, and a monolithic 70 mm diameter receiver. The narrowest trace is for one element, then follow in increasing width the traces for 2, 3, and in sequence up to 30 elements. With 30 elements, the 80% power level is retained to ±10°. By combining the relaxation on alignment due to the array, with a feedback loop, in one embodiment based on monitoring the output power of the receiver, a non-mechanical means of aligning the transmitted wave with the receiver face has been achieved. This method can be used to maximize power, or to retain a constant power level which is slightly below the most efficient operation. Hence alignment becomes a method to retain a very tight tolerance on the output power. To be effective in operation, it is necessary to have an array in two orthogonal directions, able to compensate for angular displacement along each of two axes. In another embodiment, miniature stepper motors responding to feedback information are used change the angular alignment of the transmitter with respect to the receiver.

### The Feedback Loop

The feedback loop 130 and 450 is illustrated in FIG. 2A and FIG. 2C, connecting the external controller with the implant. The basic feedback algorithm used to optimize the position of each axis of the lateral and angular alignments, and the frequency from the signal generator, is this. First, the position for each axis or the frequency is swept across its entire range with a gross step between each position or frequency. Next, the level of the receiver power output is measured at each step. Next, the position and frequency is again swept but across a smaller range centered around the best position or frequency from the previous sweep, and at a smaller step size. The process is repeated until a very fine step size thus narrowing in on the optimal frequency or position. Individual power measurements may vary due to electronic noise effects. With gross steps, it is easy to measure distinct changes, but as the step size decreases, the noise floor quickly overcomes the differences in power created by a change in position or frequency. To get a finer step size and still be able to discern a clear change in power, an averaging of ten measurements is useful. In another embodiment, the averaged measurements were filtered for each location and frequency. From digital signal processing it is known that an ideal low pass filter in the frequency domain is a sine function in the time domain. More formally, given the filter H(ω) defined below for the frequency domain

$$H ( ω ) = \{ \begin{matrix}1 , - ω \leq {ω}_{c} \leq ω \\ 0 , else ,\end{matrix}$$

the inverse discrete time/space Fourier transform h(n) of the H(ω) is equal to

$$h ( n ) = \frac{sin ( {ω}_{c} n )}{π n} ,$$

where h(n) is the impulse response of the filtering system. This particular function is known as the sine function. The output is equal to the convolution of the input with the impulse response. Since this filter is symmetric, convolution with this filter is equivalent to cross correlation. Thus, the filtered power at a particular location or frequency n₀ is

$$y ( {n}_{0} ) = {\sum }_{k = N - {n}_{0}}^{N + {n}_{0}} x ( k ) h ( k )$$

where N+1 is equally to the number of coefficients of the symmetric filter and x is the signal of measured powers. Such a filter implementation is clearly not ideal because of the finite filter length of the filter and the finite precision of the digital values; however, the power measurements are filtered only to identify a clear peak in the data. At a low angular cut off frequency of around 0.5 radians (determined empirically) most of the AC components of the power measurements are removed. By implementing this filter as part of the algorithm, an optimal position for each axis and an optimal frequency are obtained in which manual adjustments no longer yield perceivably higher powers.

### Cooling

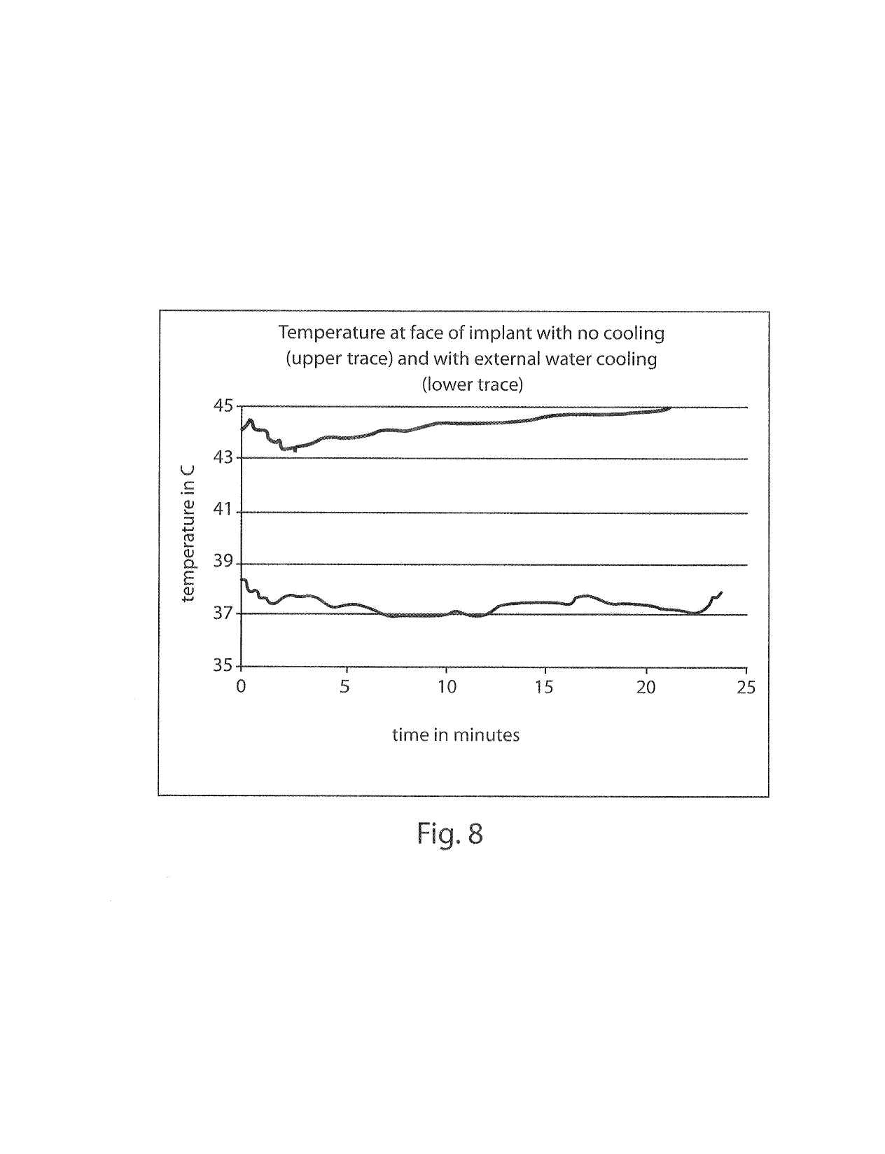

In cases where spreading the power over a large transducer area fails to meet the temperature milestone passively, a cooling method actively constrains tissue exposure to high temperature. Cooling is successfully accomplished by circulating water or another non-reactive, non-toxic liquid around the base of the transmitter assembly, and then through a heat exchanger. When the transmitter is cool enough, the temperature of the tissue between the transmitter and receiver can even be kept below ambient. The method provides cooling even to the bottom of the implant, via conduction. FIG. 8 shows the temperatures measured in a porcine test, at the top of the implant, approximately 1 cm deep into the tissue, without (upper) and with (below) external water cooling, at high ˜120 mA of charging current into a battery.

Several other embodiments accomplish the cooling goal: using a phase transition material where the transition is between 37 and 41 C or close to that range, in the transmitter and or receiver to lock the temperature at a given maximum value; using an endothermic chemical reaction in contact with the skin to absorb heat; cooling the front face of the transmitter convectively; using circuit design and elements in the receiving circuitry to increase efficiency and reduce heat given off in the implant; using transmitter transducers with side water cooling to more effectively cool the transmitter; using a boot that attaches to the skin and provides a contained water reservoir between the transmitter and the skin; using transmitter height adjustment with water or gel or gel pad coupling to reduce heat conduction to the skin; using a cooled gel pad.

### Wireless Communication System

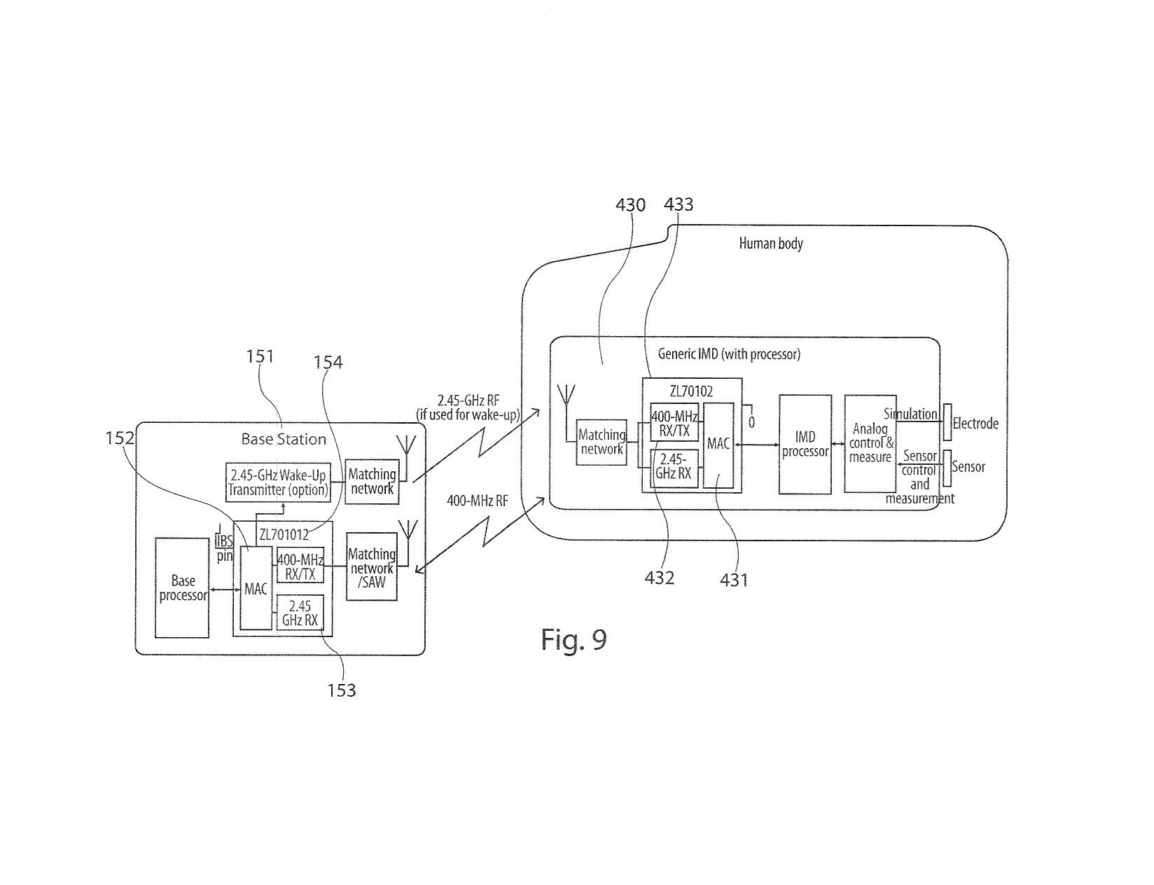

The purpose of an RF-Link is to have a wireless, bi-directional, non-invasive means of communication between a device implanted in a living human body, and an external controller. This provides the capability to remotely read out key parameters in the implant while permanently installed, and control parameters inside the implant, such as controlling a variable discharge dummy load to speed up battery discharging. FIG. 9 illustrates schematically the wireless communications RF-link 500 where an external base station 151 in an external controller 100 can communicate bi-directionally in half-duplex mode with the internal component 430 in the implant 400. The implant transceiver 430 device is paired with a microcontroller for added functionality. The base station 151 preferably is fitted with the microcontroller because sufficient power is always available. As a platform suitable for application to implants in humans, Zarlink's medical implant communications service (MICS) band transceivers ZL70102 are preferred. MICS is the industry standard for medical implants. It specifies low-power devices operating in the 400 MHz band without license requirement. Operating in the industrial, scientific and medical (ISM) band at 2.45 GHz is also license-free.

The system consists of a base station module 151, an implant module 430 and the required software package to control the system and communicate with the user interface. The hardware uses two microprocessors for the base station transceiver and two microprocessors for the implant transceiver. Zarlink provided the source code starting point, a software package that contains firmware for the microprocessors and an elaborate graphical user interface (GUI) that allows control of all features of the entire system from low-level bit addressing of registers to impedance-matching of the RF stages. The code is written in Visual C# and developed on the integrated development environment (IDE) Microsoft Visual Studio 2008.

The Zarlink chip uses a 2.45-GHz wake-up subsystem consisting of the 2.45-GHz receiver and the wake-up controller, plus an ultra-low-power, 25-kHz strobe oscillator that can be used for timing purposes. The wake-up controller is a digital subsystem that identifies when the implant module 430 receives a valid 2.45-GHz wake-up data packet from the base station 151, which is unique for a particular implant. The wake-up controller then powers up the media access controller (MAC) 431 and the 400-MHz transceiver 432, so that the implant can respond on 400 MHz and establish a two-way MICS-band link with the base station 151. While the 400-MHz link is operative, the 2.45-GHz wake-up subsystem is powered down. When the implant reverts to the sleep state, the 2.45-GHz wake-up subsystem is periodically re-enabled to listen for any possible wake-up transmissions.

In the base station 151, the MAC 152 provides a modulation signal for the external 2.45-GHz wake-up transmitter 153. The ZL70102 154 has features to facilitate and optimize a 400-MHz wake-up mode. A key feature of the ZL70102 is a fast received signal strength indicator (RSSI) sniff function that is optimized for sniffing and that leaves out operations that are required only for a normal wake-up. The bulk data communication takes place in the 400 MHz band while the wake-up calls are made in the 2.45 GHz band. The reason for the lower frequency for bulk communication is that 2.45 GHz electromagnetic waves experience significant absorption in body tissue, which is mainly water. With less loss at 400 MHz the transmitter power requirements are significantly less, an important feature for extending battery life.

When the implant 430 correctly receives the 2.45-GHz wake-up transmission from the base station 151, it responds using its 400-MHz transceiver 432. Therefore an on-chip, 2.45-GHz transmitter 152 is not needed. The base station 151 uses an external 2.45-GHz Wake-Up Transmitter module, which is controlled jointly by the application processor and the ZL70102 154. The wake-up function uses 2.45 GHz because the band is internationally designated as an ISM frequency band and so is more generally available on an international basis at a higher power level than other frequency ranges. The use of a higher transmitter power allows a reduction in the sensitivity of the wake-up receiver. Also, the use of a higher frequency tends to increase the received power available from the antenna, although this advantage is partly offset by the increased loss within the patient's body at 2.45 GHz. Taking all these factors into consideration, the overall result is a significant advantage in using 2.45 GHz. Zarlink recommends operation under the requirements for wideband data transmissions, as opposed to RFID regulations, since the allowable spectrum mask limits permit a faster rise time for the 2.45-GHz on/off keying. When operating under regulations for wideband data transmission, it may be necessary to provide frequency hopping in the 2.45-GHz transmitter 152. The bandwidth of the 2.45-GHz wake-up receiver in the ZL70102 433 is large enough that a substantial frequency spread can be used without loss of sensitivity caused by the mistuning of the input network.

1. A bio-implantable energy capture and storage assembly, comprising:
i. an acoustic energy transmitter and an acoustic energy receiver, said acoustic energy receiver also functioning as an energy converter for converting acoustic energy to electrical energy; and
ii. an electrical energy storage device connected to said energy converter, wherein said acoustic energy receiver-converter is contained within a biocompatible implant for implantation in tissue, wherein said acoustic energy transmitter is separate from said implant.

2. The bio-implantable energy capture and storage assembly of claim 1, wherein the transmitter is comprised of a 2-dimensional array of elements arranged on a support.

3. The bio-implantable energy capture and storage assembly of claim 1, wherein said substantially 2-dimensional array of elements is arranged in a circle.

4. The bio-implantable energy capture and storage assembly of claim 1, wherein said substantially 2-dimensional array of elements is arranged in a substantially regular 2-dimensional geometric shape.

5. The bio-implantable energy capture and storage assembly of claim 4, wherein said substantially regular 2-dimensional geometric shape is selected from the group consisting of a square, a pentagon, a hexagon and an octagon.

6. The bio-implantable energy capture and storage assembly of claim 1, further including a wireless feedback loop between said implant and transmitter, for monitoring one or more parameters related to an output power of the receiver.

7. The bio-implantable energy capture and storage assembly of claim 1, further including a device for cooling the energy transmitter and tissue.

8. The bio-implantable energy capture and storage assembly of claim 6, further including sensor transmitters and receivers on the acoustic energy transmitter, connected in said feedback loop.

9. The bio-implantable energy capture and storage assembly of claim 8, wherein said sensor transmitters and receivers comprise ultrasonic elements.

10. A bio-implantable energy capture and storage assembly, comprising:
iii. an acoustic energy transmitter and an acoustic energy receiver, said acoustic energy receiver also functioning as an energy converter for converting acoustic energy to electrical energy;
iv. an electrical energy storage device connected to said energy converter; and
v. a device for providing conditioned power directly to a load, connected to said energy converter, wherein said acoustic energy receiver-converter is contained within a biocompatible implant for implantation in tissue, wherein said acoustic energy transmitter is separate from said implant.

11. The bio-implantable energy capture and storage assembly of claim 10, wherein transmitter is comprised of a 2-dimensional array of elements arranged on a support.

12. The bio-implantable energy capture and storage assembly of claim 11, wherein said substantially 2-dimensional array of elements is arranged in a circle.

13. The bio-implantable energy capture and storage assembly of claim 11, wherein said substantially 2-dimensional array of elements is arranged in a substantially regular 2-dimensional geometric shape.

14. The bio-implantable energy capture and storage assembly of claim 13, wherein said substantially regular 2-dimensional geometric shape is selected from the group consisting of a square, a pentagon, a hexagon and an octagon.

15. The bio-implantable energy capture and storage assembly of claim 10, further including a wireless feedback loop between said implant and transmitter for monitoring one or more parameters related to an output power of the receiver.

16. The bio-implantable energy capture and storage assembly of claim 10, further including a method of cooling the energy transmitter and tissue.

17. The bio-implantable energy capture and storage assembly of claim 15, further including sensor transmitters and receivers on the acoustic energy transmitter, connected in said feedback loop.

18. The bio-implantable energy capture and storage assembly of claim 17, wherein said sensor transmitters and receivers comprise ultrasonic elements.

19. A process for optimizing a position of the bio-implantable energy capture and storage assembly of claim 1, which comprises positioning the assembly on a patient, measuring receiver output, repositioning the assembly and again measuring receiver output, repeating repositioning and measuring, each time repositioning the assembly a smaller amount until changes in repositioning no longer yield perceivably higher power.

20. A process for optimizing a position of the bio-implantable energy capture and storage assembly of claim 10, which comprises positioning the assembly on a patient, measuring receiver output, repositioning the assembly and again measuring receiver output, repeating repositioning and measuring, each time repositioning the assembly a smaller amount until changes in repositioning no longer yield perceivably higher power.
